(19) United States
(12) Patent Application Publication
(10) Pub. No.: US 2023/0140563 A1
(43) Pub. Date: May 4, 2023

(54) SYSTEM AND METHOD FOR THERMOELECTRIC CHARGING OF A BATTERY

(71) Applicant: Moroz Technologies Pty Ltd., GNANGARA (AU)

(72) Inventor: Paul MOROZ, GNANGARA (AU)

(21) Appl. No.: 17/912,715

(22) PCT Filed: Mar. 19, 2021
(86) PCT No.: PCT/AU2021/050258

§ 371 Date: Sep. 19, 2022

(30) Foreign Application Priority Data
Mar. 20, 2020 (AU) 2020900854

(51) Int. Cl. A61N 1/378; H10N 10/10; A61N 1/39; H01L 23/44; H02J 50/20

(52) U.S. Cl. CPC A61N 1/378; H10N 10/10; A61N 1/3975; H01L 23/44; H02J 50/20; H02J 50/15

(57) ABSTRACT

The present invention provides an implantable medical device (10), such as a neural implant, a neural stimulator, a pacemaker, a defibrillator, a glucometer or a drug pump. The device (10) includes a battery (B) providing a supply of electric power for operation of the device, and a system (1) for thermoelectric charging or re-charging of the battery (B). The system (1) includes a field-sensitive component (2) configured and/or adapted for transducing a field of magnetic energy, microwave energy, ultrasound energy, and/or X-ray energy into heat; and a thermoelectric module (4) arranged and/or connected to interface with the field-sensitive component (2) for generating an electric potential from the heat transduced by the field-sensitive component (2). The thermoelectric module (4) is arranged in electrical connection with the battery (B) for applying the electric potential to the battery (B).

### FIELD OF THE INVENTION

The invention relates to a system and method for thermoelectric charging or re-charging of a battery.

The system and method of the present invention have particular application to the field of implantable medical devices (IMDs) that are embedded within the human or animal body and rely on an electrical power supply from a battery for their operation. It will be convenient to describe the invention in this exemplary context. However, it will be appreciated that the system and method of the invention may be applicable wherever a need may arise to charge a battery deployed in difficult-to-access electronic or electro-mechanical equipment, machinery, in vehicles or even in buildings.

### BACKGROUND OF THE INVENTION

The following discussion of background is intended to enable an understanding of the present invention only. This discussion is not an acknowledgement or admission that any of the material referred to is or was part of the common general knowledge as at the date of this patent application.

Over the past decade there has been exponential growth in the use of IMDs for the treatment of humans, with over a million IMDs placed annually worldwide. Examples of such IMDs include pacemakers, defibrillators, drug pumps (e.g. for chemotherapy or for insulin), cochlear implants, peripheral and central neuro-stimulators, brain implants, joint assist and bionic devices, and spinal implants.

These devices typically rely upon an electrical power supply from lithium-based batteries, biofuels, radioisotopes, kinetic energy converters based on body movements or the piezoelectric effect, photovoltaic cells or optical charging, and inductive power transmission via paired antennas through mutual inductive coupling. Most of the power supply methods in these known devices suffer from the need for surgical procedures to replace batteries and the associated risks, costs, and inconvenience such procedures entail, and/or from achieving only a low to moderate power output.

In view of the above, it would clearly be desirable to provide a new way of (re-)-charging a battery in an IMD. More particularly, it would be desirable to provide a simple and non-invasive technique for (re-)charging a battery in an IMD implanted in a patient in a relatively fast and/or efficient manner, e.g. with a relatively high energy load.

### SUMMARY OF INVENTION

According to a broad aspect, the invention provides a system for thermoelectric charging or re-charging of a battery, especially a battery providing a supply of electric power in an implantable medical device (IMD), or a battery embedded in other difficult to access structures or equipment. The system comprises a field-sensitive component configured and/or adapted for transducing a field of one or more of magnetic energy, microwave energy, ultrasound energy, or X-ray energy into heat; and a thermo-electric module arranged and connected to interface with the field-sensitive component for generating electric potential from heat transduced by the field-sensitive component. The thermoelectric module is arranged in electrical connection with the battery, possibly switched electrical connection, for applying the electric potential to the battery.

In this way, the system of the invention is designed for charging or re-charging a battery by applying a field of magnetic energy, microwave energy, ultrasound energy, and/or X-ray energy to the field-sensitive component. In other words, no direct physical access to, or contact with, the battery is required. Rather, an externally derived energy field can be focussed and delivered safely through human tissues to re-charge a battery of an IMD without the need for an anaesthesia or surgical procedure with its associated risks and costs. The applied field of the magnetic, microwave, ultrasound, and/or X-ray energy interacts with the field-sensitive component and, via the thermoelectric module, operates to effect charging of the battery. Similarly, a battery incorporated in various types of machinery or electronic equipment or in walls of a building could be charged by the application of such an external energy field; e.g. focussed, conveyed, or channelled to an energy transducing component of a thermoelectric generator (TEG) to produce a required thermal gradient for generation of electricity, and hence wireless charging or re-charging of the battery. In this regard, the TEG can harvest the heat generated by the field-sensitive component and can then convert that heat into electricity via the Seebeck effect.

In a preferred embodiment, the thermoelectric module comprises two elements of dissimilar thermoelectric materials, especially an n-type semiconductor element (with negative charge carriers) and a p-type semiconductor element (with positive charge carriers), connected at their respective end regions, wherein at their one end region the two elements are interconnected by and/or interface with the field-sensitive component and at their opposite end region those elements are interconnected by and/or interface with a heat sink. In this way, a heat differential can be created between opposite ends of the semiconductor elements, thereby to generate the electric potential, when the energy field is applied to the field-sensitive component.

In a preferred embodiment, the field-sensitive component is configured and/or adapted to transduce at least one of alternating current magnetic field (ACMF) energy and microwave field (MWF) energy into heat. Alternatively, or in addition, the field-sensitive component may be configured and/or adapted to transduce at least one of ultrasound energy and/or X-ray energy into heat.

In a preferred embodiment, the field-sensitive component comprises or contains a plurality of particles adapted for transducing the field of magnetic energy, microwave energy, ultrasound energy, and/or X-ray energy into heat. In a particularly preferred embodiment, the field-sensitive component comprises or contains particles adapted to transduce magnetic field energy, e.g. ACMF energy and/or MWF energy into heat. The particles may comprise or contain a ferro-magnetic material or other metamaterial, such as one or more of haematite, magnetite, silicon carbide, graphite, adapted to absorb the ACMF and/or MWF energy and to transduce same into heat locally. The invention thus contemplates a thermoelectric battery (TEB) in which a thermal gradient is generated by energy-transducing particles (ETPs). The nature of the particles is such that they can transduce externally applied energy waves, such as ACMF or MWF energy, into heat which can then be converted into electricity by the Seebeck effect, hence providing for the wireless (re-)charging of a battery.

In a preferred embodiment, the particles of the field-sensitive component for transducing the energy field into heat may be particles (e.g. naked microparticles) with a diameter in the nanometre range of about 10 to about 100 nm, or microspheres (e.g. encapsulated microparticles) having a diameter in the range of about 10 to about 100 microns. Furthermore, the particles may comprise a plurality of molecular spring (MS) elements, e.g. comprising one or more of: decane, helicine or polyacetylene, and/or phase change material (PCM) such as one or more of: norbornadiene or titanium oxide. Optionally, the particles may comprise an inert shell of carboxy resin or silicon or glass that encloses or encapsulates the particle. The particles may, for example, be adapted to transduce microwave energy and/or ultrasound energy into heat.

In another alternative preferred embodiment, the field-sensitive component may comprise or contain a solid block, sheet, strip or element of material (e.g. preferably of non-particulate material) for transducing the field of energy—i.e. the magnetic energy, microwave energy, ultrasound energy, and/or X-ray energy into heat. In this context, the solid block, sheet, or element of material may once again comprise or contain a ferro-magnetic material or other metamaterial, such as one or more of haematite, magnetite, silicon carbide, or graphite, which is adapted to absorb ACMF and/or MWF energy and to transduce same into heat.

Thus, a thermoelectric generator (TEG) can be constructed with a field-sensitive component containing material (e.g. as particles) capable of transducing ACMF energy, microwave (MW) field energy and/or ultrasonic (US) field energy into heat via hysteresis loss, dielectric loss or mechanical friction, respectively. X-rays could also be applied if the TEG contained X-ray absorbing material(s) that would be heated by X-rays. This component would then generate the thermal gradient that the TEG requires to produce electricity. The heated field-sensitive component could be in physical or thermal contact with elongate and separated n-doped and p-doped semiconductor elements, opposite ends of which, in turn, are in contact with a cooler conducting material as a heat sink. Active or passive cooling and/or heat shielding of the cool side of the TEG may also be used to enhance and prolong the temperature differential with beneficial effects on the electric current produced by the TEG. Such a system would generate an electric current via the Seebeck effect for charging a battery, e.g. of an IMD or other device.

In a preferred embodiment, the thermoelectric generator (TEG) includes active or passive cooling and/or heat shielding of the cool side of the TEG. For example, the TEG may include a cooling system, e.g. preferably comprising a cooling jacket and/or a cooling circuit, at a cool side of the TEG for maintaining and/or enhancing a temperature differential to a side of the TEG heated by the field-sensitive component. In this regard, the cooling system may desirably include single-phase or two-phase immersion cooling. In a single-phase immersion cooling system, a dielectric fluid (typically a hydrocarbon or a fluorocarbon) is provided to circulate over and/or around the cool side of the TEG for effect heat exchange. In a two-phase cooling system, a dielectric liquid may be used to absorb heat from the cool side of the TEG which, in turn, causes the liquid to evaporate into a vapour thereby conducting heat away from the cool side. When the vapour cools and loses heat as it circulates away from the cool side of the TEG, it re-condenses into a liquid for circulating back to the cool side. The enhancement and/or maintenance of a high temperature differential between the heated and cool sides of the TEG supports or drives the Seebeck effect and promotes reliable current generation and effective battery charging. The heat shielding proposed for the cool side of the TEG may include the use of vanadium dioxide or organic polymers. Alternatively, or in addition, the heat shielding may employ spatial distancing between the heated side and the cool side of the TEG to reduce the prospect of the cool side being inadvertently heated by the incident field energy or via heat transfer (e.g. conduction) from the heated side.

In a preferred embodiment, the thermoelectric generator (TEG) is configured so that the field-sensitive component is located physically spaced from the cool side of the TEG. In this regard, the spacing is designed to provide thermal shielding and to reduce prospects of heat transfer (e.g. conduction) or field energy application to the cool side of the TEG. For example, physical separation or spacing of the field-sensitive component from the cool side of the TEG may be achieved by designing a selected geometry of the thermoelectric materials, e.g. n-type and p-type semiconductor elements, of the TEG.

A thermoelectric battery in a human IMD may typically rely upon a temperature difference of up to about 8° C. between a deep tissue location and a subcutaneous location. The energy-transducing particles (ETPs) in an externally applied energy field according to the invention could safely generate thermal gradients several times greater in a correctly designed TEG, resulting in a significantly greater energy loading to an IMD battery. For example, milligram ranges of ferromagnetic particles in a radiofrequency magnetic field up to 400 Oersted can readily produce a heat energy output of 30 Watts for as long as the field is applied. Externally applied MWF systems, such as phased annular arrays, can safely introduce or apply 10-30 Watts to deep tissue locations. High frequency focussed ultrasound can safely apply about 100 Watts per square cm of deep tissue for several seconds.

In a preferred embodiment, the system for thermoelectric charging of the battery includes a field generator for generating a field of magnetic energy, microwave energy, ultrasound energy and/or X-ray energy and for applying that field to the field-sensitive component, and especially to a patient within whom an IMD having a battery for supply of electric power to the IMD and a system for thermoelectric charging of the battery has been implanted. The field generator preferably has an applicator unit for focussed or channelled application of the respective energy field to the field-sensitive component of the system in a thermoelectric generator (TEG) that is connected to, or integrated with, the battery in the IMD.

According to another aspect, the invention provides an implantable medical device (IMD) comprising: a battery for providing a supply of electric power for operation of the device, and a system for thermoelectric charging of the battery according to any one of the embodiments described above. Examples of the IMD device include a neural implant, a neural stimulator, a pacemaker, a defibrillator, a glucometer, or a drug pump.

The re-charging process would thus involve a patient who a priori has an IMD in place and incorporating a TEG (optionally containing ETPs), presenting to a centre with an external energy field generator and applicator unit and then being exposed to the energy field for an appropriate period of time while the re-charging occurs. Preferably, this would not require any anaesthesia or surgical procedure and could occur as often as necessary to ensure a consistently strong battery output. The risk and the cost (i.e. both financial cost and time cost) to the patient and the health system for such battery re-charging would be greatly reduced.

According to a further aspect, the invention provides a method of charging or re-charging of a battery, especially a battery that supplies electric power in an implantable medical device (IMD). The method comprises steps of: applying a field of magnetic energy, microwave energy, ultrasound energy, and/or X-ray energy to a field-sensitive component adapted for transducing that energy field into heat; generating an electric potential from heat transduced by the field-sensitive component via a thermoelectric module arranged and/or connected to interface with the field-sensitive component; and applying that electric potential to the battery to effect charging or re-charging, typically by electrically connecting an output of the thermoelectric module with the battery.

In a preferred embodiment, as noted above, the invention employs particles to transform or transduce magnetic and/or microwave energy into heat by hysteresis and/or dielectric loss effects. Thus, the method of the invention preferably involves applying an alternating current magnetic field (ACMF) and/or a microwave field (MWF) to the particles contained or comprised in the field sensitive component. A pattern of heat output from the particles can be modulated or attenuated by incorporation of molecular springs (MS) and phase change materials (PCM) into the particles. As also noted above, the particles will typically comprise some combination of haematite, magnetite, silicon carbide and graphite—all of which can transduce ACMF, MW and US energy to varying degrees. The size of the particles will be determined by an optimum set of energy wavelengths and frequencies to produce the best heating. Additives such as phase change materials or molecular springs may be incorporated in certain ways to facilitate heating patterns in certain circumstances. Other metals, metalloids, or non-metals may be incorporated into the particles to optimize energy output. In the case of X-ray transduction, particles that could absorb an X-ray bolus would likely be of similar or the same composition as the metal from which the X-rays were generated to optimise heating.

In a preferred embodiment, the method of the invention involves designing the field-sensitive component and providing or arranging the field-sensitive component in a position and/or orientation to receive the applied field of magnetic energy, microwave energy, ultrasound energy, or X-ray energy as effectively and as efficiently as possible. In this regard, the system of the invention may need to be implanted spaced at a distance from the functional aspect of the IMD and connected to it by electrical wires so that the field-sensitive component of the system may be located more favourably for application of the energy field. In this regard, it will be noted that any one of magnetic energy, microwave energy, ultrasound energy, or X-ray energy can present health risks to a patient if applied in excessive doses or intensities. Furthermore, excessive heating at the field-sensitive component could cause damage to other components of the IMD.

In a preferred embodiment, a magnetron would be used to generate MW energy which could be focussed onto the field-sensitive component of the system by a single beam applicator head on a robotic arm or by a phased microwave array if the IMD were implanted in the torso or brain of a patient. A conducting funnel could channel an MWF onto the field-sensitive component also deep within machinery or electronic equipment. Handheld or robotic arm-mounted high-frequency ultrasound (US) applicators (100W) could also deliver energy to an implanted field-sensitive component of the system by a single beam or alternatively a phased array could be employed. A US conducting rod could be inserted into electronic hardware or machinery for direct contact with the field-sensitive component of the system embedded deep within the device to deliver the US energy to its energy transducing particles. Currently, robotic arms can be employed to deliver precise high doses of X-rays (External Beam Radiation Therapy) to deep tissue targets. Such an approach could be used to apply X-ray energy onto an X-ray absorbing field-sensitive component in a system of the invention.

### BRIEF DESCRIPTION OF THE DRAWINGS

For a more complete understanding of the invention and advantages thereof, exemplary embodiments of the invention are explained in more detail in the following description with reference to the accompanying drawing figures, in which like reference signs designate like parts and in which:

FIG. 1 is a schematic side view of a system according to a preferred embodiment of the invention, showing application of ACMF and/or MWF energy;

FIG. 2 is a schematic side view of a system according to another embodiment of the invention, showing application of field energy;

FIG. 3 is a schematic side view of a system according to a further embodiment of the invention, showing application of field energy;

FIG. 4 is a schematic side view of a system according to yet another embodiment of the invention, showing application of field energy; and

FIG. 5 is a flow diagram that schematically represents a method of charging a battery in an IMD according to an embodiment of the invention.

The accompanying drawings are included to provide a further understanding of the present invention and are incorporated in and constitute a part of this specification. The drawings illustrate particular embodiments of the invention and together with the description serve to explain the principles of the invention. Other embodiments of the invention and many of the attendant advantages will be readily appreciated as they become better understood with reference to the following detailed description.

It will be appreciated that common and/or well understood elements that may be useful or necessary in a commercially feasible embodiment are not necessarily depicted in order to facilitate a more abstracted view of the embodiments. The elements of the drawings are not necessarily illustrated to scale relative to each other. It will also be understood that certain actions and/or steps in an embodiment of a method may be described or depicted in a particular order of occurrences while those skilled in the art will understand that such specificity with respect to sequence is not actually required.

### DETAILED DESCRIPTION OF THE EMBODIMENTS

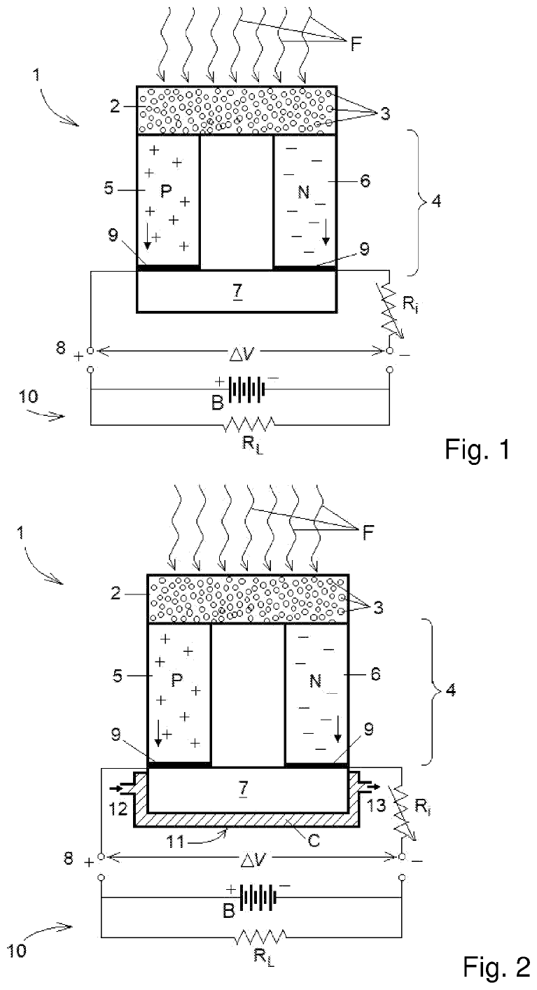

With reference firstly to FIG. 1 of the drawings, a system 1 for wireless charging and recharging a battery B in an implantable medical device 10 is shown schematically. The system 1 comprises a field-sensitive component 2 configured and/or adapted for transducing a field F of applied energy, especially radio-frequency alternating current magnetic field (ACMF) energy, and/or microwave field (MWF) energy into heat. In this regard, the field-sensitive component 2 contains a dense packed array of ferro-magnetic particles 3 adapted for transducing ACMF energy and/or MWF energy into heat.

The system 1 furthermore comprises a thermoelectric module 4 arranged and connected to interface with the field-sensitive component 2 for generating an electric potential ΔV from heat transduced by the particles 3 of the field-sensitive component 2. The thermoelectric module 4 comprises a thermoelectric element 5 of a p-type semi-conductor material having positive charge carriers, and a thermoelectric element 6 of an n-type semiconductor material having negative charge carriers. Both the thermoelectric elements 5, 6 are connected (i.e. thermally conductively and preferably also physically) at one of their respective end regions with the field-sensitive component 2. In particular, at their one end region, both the n-type and p-type semiconductor elements 5, 6 are interconnected by and interface with the field-sensitive component 2 and, at an opposite end region, those semiconductor elements 5, 6 are interconnected by and interface with a heat sink 7. In this way, a temperature differential is created between the opposite end regions of the thermo-electric elements 5, 6 to generate the electric potential ΔV when the magnetic or micro-wave energy field F is applied to the field-sensitive component 2. The thermoelectric module 4 will usually include some internal resistance Rᵢ (optionally a variable resistance) and is arranged for electrical connection with the battery B at an output 8, preferably via a switchable electrical connection (not shown), for applying the electric potential ΔV to the battery B. It will be noted that, while the end region of the n-type and p-type thermoelectric elements 5, 6 are in thermally conductive connection or contact with the heat sink 7, those end regions are electrically isolated from the heat sink 7, e.g. via electrically insulating end sheets or pads 9, and electrically connected separately, e.g. via circuit wiring, to the output 8.

The nature of the particles 3 is such that they can transduce externally applied energy waves in an ACMF or MWF into heat to generate the thermal gradient which can then be converted into electricity by the Seebeck effect, hence producing a wireless re-charging of the battery. The heated field-sensitive component 2 would need to be well thermally insulated from the patient's body tissues to prevent thermal injury. To this end, thermal shielding may be employed. The system 1 is designed to generate an electric current via the Seebeck effect that can then be used to charge the contacted battery B in the IMD 10, as shown in FIG. 1. In this context, it will be noted that a resistance R_{L} is just a simplified representation of the operational load applied to the battery B within the IMD 10. Further innovations such as shielding, insulation, spatial rearrangement, and/or incorporation of new materials, such as conducting polymers or metamaterials, into system 1, battery B and/or implanted device 10 to facilitate safe and effective re-charging are contemplated, as will be described later.

This embodiment of the invention relies on the application of a radiofrequency alternating current magnetic field, a microwave field, and/or an ultrasonic field to deliver energy specifically to the particles 3 in the implanted thermoelectric generator (TEG) of system 1. These energy fields F should be focussed as much as possible onto the field-sensitive component 2 to minimise collateral tissue damage by extraneous energy; for example, Joule heating of normal tissue by an ACMF. A number of electromagnetic coil geometries could be used to produce a uniform ACMF that encompasses an implanted TEG, including Helmholtz and Maxwell coils, pancake coils of various geometries, a simple solenoid or electromagnetic coils having soft iron cores wound with copper wire, such as a Halbach array. The configuration of the ACMF applicator would depend on the location of the TEG system 1. For example, if the TEG system 1 were located in an arm of the patient and connected by wiring to the IMD elsewhere in the body, a simple solenoid applicator head of a field generator (not shown) could accommodate the arm and apply an ACMF safely. If the TEG system 1 were located in the thorax or abdomen of the patient, then pancake coils in an applicator head of a field generator (not shown) sited above and below the supine patient could apply the ACMF.

Referring now to FIG. 2 of the drawings, a system 1 for wireless charging and/or recharging a battery B in an implantable medical device or other device 10 to be located in an inaccessible position is shown schematically. The system 1 of this embodiment is very similar to the system of FIG. 1 with a field-sensitive component 2 configured and/or adapted for transducing a field F of applied energy, such as ACMF energy and/or MWF energy, into heat. To this end, the field-sensitive component 2 contains densely packed ferro-magnetic material or particles 3 for transducing ACMF energy and/or MWF energy into heat. The system 1 furthermore includes a thermoelectric module (TEG) 4 arranged and connected interfacing with the field-sensitive component 2 for generating an electric potential ΔV from heat transduced by the material 3 of the field-sensitive component 2. To this end, the thermoelectric module 4 has a thermoelectric element 5 of p-type semi-conductor material with positive charge carriers, and a thermoelectric element 6 of n-type semiconductor material with negative charge carriers. The thermoelectric elements 5, 6 are elongate and are connected thermally, and preferably also physically, at one of their respective end regions with the field-sensitive component 2. At their opposite end regions, those semiconductor elements 5, 6 are interconnected by and interface with a heat sink 7 for creating a temperature differential between opposite end regions of the thermo-electric elements 5, 6 to generate an electric potential ΔV when the energy field F is applied to the field-sensitive component 2. A difference with this embodiment is a cooling arrangement 11 provided at the heat sink 7. In particular, the heat sink 7 has an active cooling system that includes a cooling jacket or cooling circuit 11 for circulating a coolant C to the end regions of the n-type and p-type semiconductor elements 5, 6; for example, via an inlet 12 and an outlet 13 of the cooling jacket 11. In this regard, the coolant C could be a dielectric liquid which is introduced at inlet 12 and absorbs heat from the end regions of the n-type and p-type semiconductor elements 5, 6 at the heat sink 7. This causes the liquid to change phase into a vapour conducting heat away from the elements 5, 6. When the vapour cools and loses heat as it circulates away from the outlet 13 of the TEG 4, it re-condenses into a liquid for circulating back to the inlet 12.

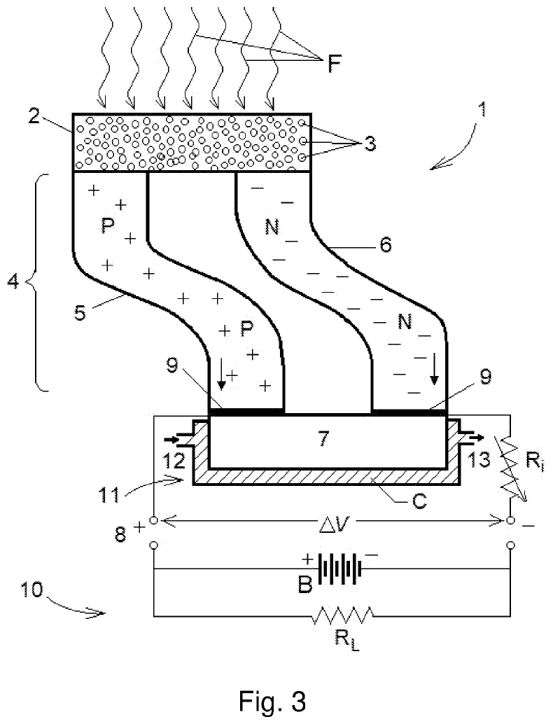

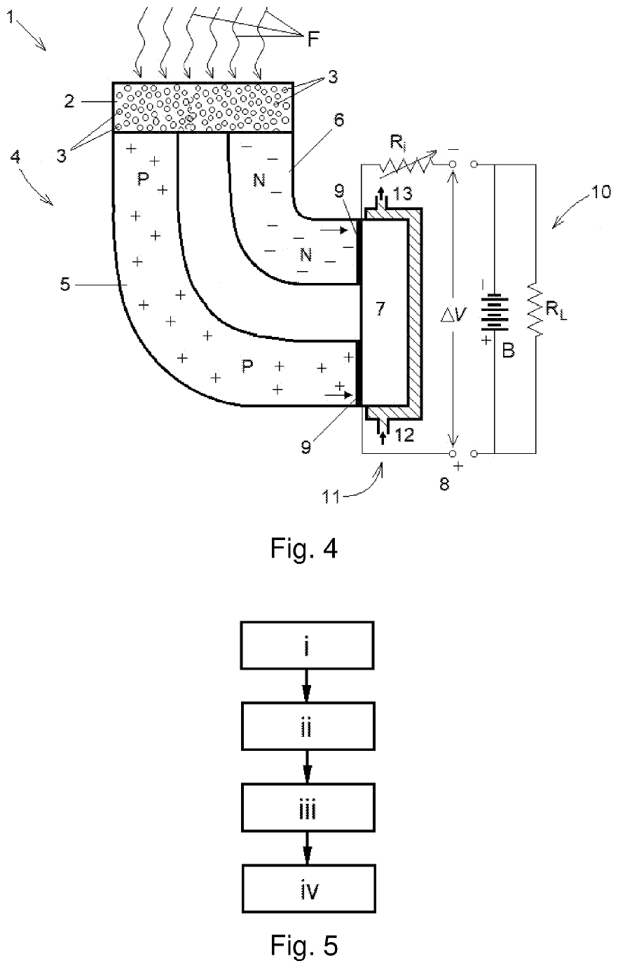

Referring to FIGS. 3 and 4 of the drawings, further embodiments of a system 1 for wireless charging and/or recharging a battery B in an implantable medical device or other device 10 designed/intended for an inaccessible location is shown schematically. The system 1 of these embodiments is very similar to the system of FIG. 2 and again includes an active cooling arrangement 11 at the heat sink 7. These two embodiments illustrate schematically the manner in which the system 1 may be configured such that the field-sensitive component 2 is located physically spaced from the cool side of the TEG 4—i.e. from the end regions of the n-type and p-type semiconductor elements 5, 6 at the heat sink 7. To this end, the elongate semiconductor elements 5, 6 of the TEG 4 are designed with a geometry selected to provide a spacing or separation between both an area of incidence of the field energy F to the field-sensitive component 2 and thermal shielding or isolation of the cool side of the TEG 4—i.e. of the end regions of the n-type and p-type semiconductor elements 5, 6 at the heat sink 7. In this way, the chances of an inadvertent and undesired heat transfer to the end regions of the n-type and p-type semiconductor elements 5, 6 at the heat sink 7 can be reduced or minimised.

With reference to FIG. 5 of the drawings, a flow diagram is shown to illustrate schematically the steps in a method of charging a battery according to the invention using the system 1 of the embodiments described above with reference to any one of FIGS. 1 to 4. In this example, the invention is employed in an implantable medical device (IMD) 10, such as a neural implant, a pacemaker, a defibrillator, a glucometer, or a drug pump. The IMD 10 has a battery B that provides a supply of electric power for operation of the IMD. In this regard, the first box i of FIG. 5 represents the step of arranging a field generator adjacent to and/or around a body of the patient in which the IMD is implanted. The field generator may comprise an array of coils or electromagnets for generating an electromagnetic field, especially an alternating current magnetic field (ACMF), and/or a magnetron for generating a microwave energy field (MWF). The second box ii of FIG. 5 represents a step of activating the field generator to apply the ACMF energy and/or the MWF energy to the field-sensitive component 2 of the system 1 in the patient's body, whereby the material or particles 3 packed in that component 2 transform or transduce that energy field F into heat. The third box iii of FIG. 5 represents the step of generating an electric potential ΔV from heat transduced by the field-sensitive component 2 via the thermoelectric module 4 connected to interface with the field-sensitive component 2. The final box iv in FIG. 5 of the drawings represents the step of applying that electric potential ΔV to the battery B to effect the charging or re-charging, typically by electrically connecting an output 8 of the thermoelectric module 4 with the battery B, for example by switching (not shown).

Although specific embodiments of the invention are illustrated and described herein, it will be appreciated by persons of ordinary skill in the art that a variety of alternative and/or equivalent implementations exist. It should be appreciated that each exemplary embodiment is an example only and is not intended to limit the scope, applicability, or configuration in any way. Rather, the foregoing summary and detailed description will provide those skilled in the art with a convenient road map for implementing at least one exemplary embodiment, it being understood that various changes may be made in the function and arrangement of elements described in an exemplary embodiment without departing from the scope as set forth in the appended claims and their legal equivalents. Generally, this application is intended to cover any adaptations or variations of the specific embodiments discussed herein.

It will also be appreciated that the terms “comprise”, “comprising”, “include”, “including”, “contain”, “containing”, “have”, “having”, and any variations thereof, used in this document are intended to be understood in an inclusive (i.e. non-exclusive) sense, such that the process, method, device, apparatus, or system described herein is not limited to those features, integers, parts, elements, or steps recited but may include other features, integers, parts, elements, or steps not expressly listed and/or inherent to such process, method, device, apparatus, or system. Furthermore, the terms “a” and “an” used herein are intended to be understood as meaning one or more unless explicitly stated otherwise. Moreover, the terms “first”, “second”, “third”, etc. are used merely as labels, and are not intended to impose numerical requirements on or to establish a certain ranking of importance of their objects. In addition, reference to positional terms, such as “lower” and “upper”, used in the above description are to be taken in context of the embodiments depicted in the figures, and are not to be taken as limiting the invention to the literal interpretation of the term but rather as would be understood by the skilled addressee in the appropriate context.

1. An implantable medical device, such as a neural implant, a neural stimulator, a pacemaker, a defibrillator, a glucometer or a drug pump, the device comprising: a battery providing a supply of electrical power for operation of the device, and a system for thermoelectric charging or re-charging of the battery, the system comprising:
a field-sensitive component configured and/or adapted for transducing a field of magnetic energy, microwave energy, ultrasound energy, and/or X-ray energy into heat; and
a thermoelectric module arranged and/or connected to interface with the field-sensitive component for generating an electric potential from the heat transduced by the field-sensitive component;
wherein the thermoelectric module is arranged in electrical connection with the battery for applying the electric potential to the battery.

2. A device according to claim 1, wherein the field-sensitive component is adapted for transducing one or more of alternating current magnetic field (ACMF) energy, and microwave field (MWF) energy into heat.

3. A device according to claim 2, wherein the field-sensitive component comprises a plurality of particles adapted for transducing ACMF energy, and/or the MWF energy into heat.

4. A device according to claim 2, wherein the particles comprise or contain a ferro-magnetic material or one or more of haematite, magnetite, silicon carbide, graphite, for absorbing ACMF and/or MWF energy to be transduced into heat locally.

5. A device according to claim 1, wherein the particles comprise microparticles having a diameter in the range of about 10 microns to about 100 microns, and/or nanoparticles with a diameter in the nanometer range.

6. A device according to claim 1, wherein the particles comprise a plurality of molecular spring (MS) elements, including one or more of: decane, helicine or polyacetylene; and/or phase change materials (PCM), including one or more of: norbornadiene or titanium oxide.

7. A device according to claim 3, wherein the particles comprise an inert shell of resin or silicon or glass to enclose or encapsulate the particle.

8. A device according to claim 1, wherein the field-sensitive component comprises or contains a solid block, sheet, strip, or element of material for transducing the field of magnetic energy, micro-wave energy, ultrasound energy, and/or X-ray energy into heat, wherein the material is selected from the group comprising haematite, magnetite, silicon carbide, and graphite.

9. A device according to claim 1, wherein the thermoelectric module comprises two elements of dissimilar thermoelectric material, especially an n-type semiconductor element and a p-type semiconductor element, connected at their respective end regions, wherein at one end region the two elements are interconnected by and/or interface with the field-sensitive component and at an opposite end region the two elements are interconnected by and/or interface with a heat sink.

10. A device according to claim 1, wherein the thermoelectric module includes a cooling system, preferably including a cooling jacket and/or a cooling circuit, for maintaining and/or enhancing a temperature differential with respect to a side of the thermoelectric module heated by the field-sensitive component.

11. A device according to claim 10, wherein the cooling system includes a circuit for a coolant, wherein the cooling circuit provides single-phase or two-phase immersion cooling.

12. A device according to claim 10, wherein the active cooling system forms the heat sink at the opposite end region of the two elements of dissimilar thermoelectric material, especially an n-type semiconductor element and a p-type semiconductor element.

13. A device according to claim 1, wherein the thermoelectric module includes heat shielding for maintaining and/or enhancing a temperature differential to a side of the thermo-electric module heated by the field-sensitive component.

14. A device according to claim 13, wherein the heat shielding comprises locating the heated side of the thermoelectric module remote from the cool side.

15. An implantable medical device, such as a pacemaker, a defibrillator, or a drug pump, the device comprising: a battery providing a supply of electric power for operation of the device, and a system for thermoelectric charging or re-charging of the battery, the system comprising:
a field-sensitive component configured and/or adapted for transducing a field of magnetic energy, microwave energy or ultrasound energy into heat; and
a thermoelectric module arranged and/or connected to interface with the field-sensitive component for generating an electric potential from the heat transduced by the field-sensitive component, the thermoelectric module having a cooling system for maintaining a temperature differential with respect to a side of the module heated by the field-sensitive component;
wherein the thermoelectric module is arranged in electrical connection with the battery for applying the electric potential to the battery.

16. (canceled)

17. A system for thermoelectric charging or re-charging of a battery in a device for deployment in an inaccessible location, such as an implantable medical device, the battery providing a supply of electrical power for operation of the device, the system comprising:
a field-sensitive component configured and/or adapted for transducing a field of magnetic energy, microwave energy, ultrasound energy, and/or X-ray energy into heat; and
a thermoelectric module arranged and/or connected to interface with the field-sensitive component for generating an electrical potential from the heat transduced by the field-sensitive component;
wherein the thermoelectric module is arranged in electrical connection with the battery for applying the electrical potential to the battery.

18. A system according to claim 17, wherein the field-sensitive component is for transducing one or more of alternating current magnetic field (ACMF) energy and microwave field (MWF) energy into heat.

19. A system according to claim 18, wherein the field-sensitive component is comprised of a material adapted for transducing ACMF energy and/or the MWF energy into heat, the material comprising a plurality of particles suited or adapted for transducing ACMF energy and/or the MWF energy into heat.

20. A system according to claim 19, wherein the particles comprise or contain ferro-magnetic material or other metamaterials, especially one or more of haematite, magnetite, silicon carbide, graphite, for absorbing ACMF and/or MWF energy to be transduced into heat.

21. A system according to claim 19, wherein the particles comprise microparticles having a diameter in the range of about 10 microns to about 100 microns, and/or naked microparticles or nanoparticles with a diameter in the nanometer range.

22. A system according to claim 19, wherein the particles comprise a plurality of molecular spring (MS) elements, including one or more of: decane, helicine or polyacetylene; and/or phase change materials (PCM), including one or more of: norbornadiene or titanium oxide.

23. A system according to claim 17, wherein the field-sensitive component comprises or contains a solid block, sheet, strip, or element of material adapted for transducing the field of magnetic energy, micro-wave energy, ultrasound energy, and/or X-ray energy into heat, wherein the material is selected from the group comprising haematite, magnetite, silicon carbide, and graphite.

24. A system according to claim 17, wherein the thermoelectric module includes an n-type semiconductor element and a p-type semiconductor element connected at their respective end regions, wherein at one end region the two elements are interconnected by and/or interface with the field-sensitive component and at an opposite end region the two elements are interconnected by and/or interface with a heat sink.

25. A system according to claim 17, wherein the thermoelectric module includes an active cooling system, including a cooling jacket or a cooling circuit, for maintaining and/or enhancing a temperature differential to a side of the thermoelectric module heated by the field-sensitive component.

26. A system according to claim 25, wherein the active cooling system forms a heat sink at the end region of the n-type and p-type semiconductor elements.
